(19) United States
(12) Patent Application Publication
(10) Pub. No.: US 2007/0067926 A1
(43) Pub. Date: Mar. 29, 2007

(54) Agent for dyeing keratin fibers containing 2-amino-5-methyl-phenol and m-aminophenoles

(76) Inventors: Manfred Schmitt, Bensheim-Auerbach (DE); Gabricle de Waal, Gross-Umstadt (DE); Annelie Dohling, Munster (DE); Andreas Pfefferkorn, Mannheim (DE)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/524,138

(22) Filed: Sep. 20, 2006

(30) Foreign Application Priority Data
Sep. 21, 2005 (DE) 202005014897.0

(51) Int. Cl. A61K 8/00

(52) U.S. Cl. 8/405

(57) ABSTRACT

The present patent application relates to an agent for oxidative coloring of keratin fibers, containing a combination of 2-amino-5-ethylphenol and at least one m-aminophenol of general Formula (I) 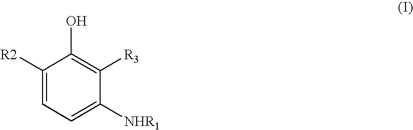
wherein R1=H or C₂H₄OH; R2=CH₃ or C₂H₅, and R3=H or CH₃;
wherein R2 is not equal to CH₃, if R1 and R3 equal H; or physiologically compatible, water-soluble salts thereof.

### FIELD OF THE INVENTION

The present invention relates to an agent for oxidative coloring of keratin fibers, particularly human hair, containing a combination of 2-amino-5-methyl-phenol and certain m-aminophenoles of general Formula (I) as color components.

### BACKGROUND OF THE INVENTION

In the area of coloring of keratin fibers, especially the coloring of hair, oxidative dyes have acquired a substantial significance. Coloring takes place through the reaction of certain developer substances with certain coupler substances in the presence of a suitable oxidizing agent. The following can be used as developer substances in particular: 2,5-diaminotoluene, 2,5-diaminophenyl ethanol, p-aminophenol, 1,4-diaminobenzene, and 4,5-diamino-1-(2-hydroxyethyl)pyrazole, whereas, for example, resorcinol, 2-methyl resorcinol, 1-naphthol, 3-aminophenol, m-phenylenediamine, 2-amino-4-(2′-hydroxyethyl)amino anisole, 1,3-diamino-4-(2′-hydroxyethoxy)benzene, and 2,4-diamino-5-fluorotoluene can be mentioned as coupler substances.

In addition to stability of hair colors for at least 4 to 6 weeks, numerous additional requirements are placed on oxidative dyes used for coloring human hair. Thus, the dyes must be harmless with respect to toxicology and dermatology, and the targeted hair colorings should exhibit good lightfastness, permanent wave fastness, fastness to rubbing, and stability with respect to shampooing as well as sufficient resistance with respect to sweat secretions. An additional requirement is that it should be possible to produce a broad palette of different color shades by combining suitable developer substances and coupler substances.

One particular problem when shading color tones is the even uptake of dye from the hair roots all the way to the hair tips as well as the shine of the colored hair. The use of direct-penetrating, yellow-coloring aromatic nitro dyes together with oxidative hair colorant precursors can solve the problem described with the shine to some extent; however, the problem with color balance from the roots to the tips is not significantly improved.

A solution to this problem can be found in German Patent No. DE 28 33 989 A1 in which the use of 2-amino-5-methylphenol was suggested as an oxidative yellow-coloring dye in oxidative hair colorants. This compound is well-suited as a shading dye to create light blonde tones and golden tones; however, it cannot fully meet the requirements especially with respect to the aforementioned even uptake of dye from the hair roots all the way to the hair tips. Particularly the area of the previously colored hair is frequently significantly darker than the roots. A person skilled in the art calls this beam formation.

Furthermore, German Patent No. DE 102 51 830 A1 suggests the use of 2-amino-5-ethylphenol as an oxidative dye in oxidative hair colorants to solve the problem described.

### SUMMARY OF THE INVENTION

Surprisingly, it was found that when a combination of 2-amino-5-ethylphenol with m-aminophenoles of general Formula (I) is used, the requirements placed on oxidative dyes for keratin fibers, particularly in relation to the prevention of the described matte formation, are fulfilled to a particularly great extent.

### DETAILED DESCRIPTION OF THE INVENTION

The object of the present invention is thus an agent for oxidative coloring of keratin fibers such as, for example, wool, fur, feathers, or hair, and especially human hair, with said agent characterized in that it contains a combination of 2-amino-5-ethylphenol and at least one m-aminophenol of general Formula (I), 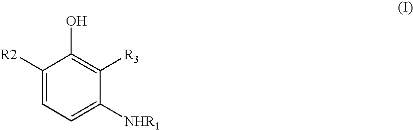
wherein R1=H or C₂H₄OH; R2=CH₃ or C₂H₅, and R3=H or CH₃;
wherein R2 is not equal to CH₃, if R1 and R3 equal H;
   or physiologically compatible, water-soluble salts thereof.

The 2-amino-5-ethylphenol and the m-aminophenoles of general Formula (I) can be used either as a free base or in the form of their physiologically compatible salts with inorganic or organic acids such as, for example, hydrochloric acid, sulfuric acid, phosphoric acid, acetic acid, propionic acid, lactic acid, or citric acid.

Preferred m-aminophenoles of general Formula (I) are 3-amino-2,6-dimethylphenol, 5-((2-hydroxyethyl)amino)-2-methylphenol, and 5-amino-2-ethylphenol or their physiologically compatible salts with inorganic or organic acids such as, for example, hydrochloric acid, sulfuric acid, phosphoric acid, acetic acid, propionic acid, lactic acid, or citric acid.

The 2-amino-5-ethylphenol or its physiologically compatible salts with inorganic or organic acids such as, for example, hydrochloric acid, sulfuric acid, phosphoric acid, acetic acid, propionic acid, lactic acid, or citric acid are contained in the dye according to the invention in a total quantity of about 0.001% to 5% by weight, wherein a quantity of about 0.001% to 2% by weight and particularly of from 0.005% to 1% by weight is preferred.

The m-aminophenoles of general Formula (I) or their physiologically compatible salts are contained in the dye according to the invention in a total quantity of about 0.001% to 5% by weight, wherein a quantity of about 0.001% to 4% by weight and particularly of from 0.005% to 3% by weight is preferred.

To obtain additional color shades, other typical oxidative dyes, for example developer substances or coupler substances, can be added, either alone or in a mixture with one another, wherein these developer substances and coupler substances can be present in the form of free bases or as physiologically compatible salts with inorganic or organic acids such as, for example, hydrochloric acid, sulfuric acid, phosphoric acid, acetic acid, propionic acid, lactic acid, or citric acid.

Examples of preferred developer substances that can be considered include 1,4-diaminobenzene (p-phenylenediamine), 1,4-diamino-2-methylbenzene (p-toluylenediamine), 1,4-diamino-2,6-dimethylbenzene, 1,4-diamino-3,5-diethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 4-(2,5-diaminophenyl)-2-((diethylamino)methyl)thiophene, 2-chloro-3-(2,5-diaminophenyl)thiophene, 1,4-diamino-2-(pyridine-3-yl)benzene, 2,5-diaminobiphenyl, 2,5-diamino-4′-(1-methylethyl)-1,1′-biphenyl, 2,3′,5-triamino-1,1′-biphenyl, 1,4-diamino-2-(methoxymethyl)benzene, 1,4-diamino-2-aminomethylbenzene, 1,4-diamino-2-((phenylamino)methyl)benzene, 1,4-diamino-2-((ethyl-(2-hydroxyethyl)amino)methyl)benzene, 1,4-diamino-2-hydroxymethylbenzene, 1,4-diamino-2-(2-hydroxyethoxy)benzene, 2-(2-(acetylamino)ethoxy)-1,4-diaminobenzene, 4-phenylaminoaniline, 4-dimethylaminoaniline, 4-diethylaminoaniline, 4-dipropylaminoaniline, 4-[ethyl(2-hydroxyethyl)amino]aniline, 4-[di-(2-hydroxyethyl)amino]aniline, 4-[di-(2-hydroxyethyl)amino]-2-methylaniline, 4-[(2-methoxyethyl)amino]aniline, 4-[(3-hydroxypropyl)amino]aniline, 4-[(2,3-dihydroxypropyl)amino]aniline, 4-(((4-aminophenyl)methyl)amino)aniline, 4-[(4-aminophenylamino)methyl]phenol, 1,4-diamino-N-(4-pyrrolidin-1-ylbenzyl)benzene, 1,4-diamino-N-furan-3-ylmethylbenzene, 1,4-diamino-N-thiophen-2-ylmethylbenzene, 1,4-diamino-N-furan-2-ylmethylbenzene, 1,4-diamino-N-thiophen-3-ylmethylbenzene, 1,4-diamino-N-benzylbenzene, 1,4-diamino-2-(1-hydroxyethyl)benzene, 1,4-diamino-2-(2-hydroxyethyl)benzene, 1,4-diamino-2-(1-methylethyl)benzene, 1,3-bis-[(4-aminophenyl)(2-hydroxyethyl)amino]-2-propanol, 1,4-bis-[(4-aminophenyl)amino]butane, 1,8-bis-(2,5-diaminophenoxy)-3,6-dioxaoctane, 2,5-diamino-4′-hydroxy-1,1′-biphenyl, 2,5-diamino-2′-trifluoromethyl-1,1′-biphenyl, 2,4′,5-triamino-1,1′-biphenyl, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetraaminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 4,5-diamino-1-pentyl-1H-pyrazole, 4,5-diamino-1-(phenylmethyl)-1H-pyrazole, 4,5-diamino-1-((4-methoxyphenyl)methyl-1H-pyrazole, 2-aminophenol, 2-amino-6-methylphenol, 2-amino-5-methylphenol, 1,2,4-trihydroxybenzene, 2,4-diaminophenol, 1,4-dihydroxybenzene, and 2-(((4-aminophenyl)amino)methyl)-1,4-diaminobenzene.

Examples of particular coupler substances are N-(3-dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di-[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino-4-(2,3-dihydroxypropoxy)benzene, 1,3-diamino-4-(3-hydroxypropoxy)benzene, 1,3-diamino-4-(2-methoxyethoxy)benzene, 2,4-diamino-1,5-di(2-hydroxyethoxy)benzene, 1-(2-aminoethoxy)-2,4-diaminobenzene, 2-amino-1-(2-hydroxyethoxy)-4-methylamino benzene, 2,4-diaminophenoxy acetic acid, 3-[di(2-hydroxyethyl)amino]aniline, 4-amino-2-di[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)methane, 1,3-diamino-2,4-dimethoxybenzene, 2,6-bis(2-hydroxyethyl)aminotoluene, 4-hydroxyindole, 3-dimethylamino phenol, 3-diethylamino phenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxyphenyl)amino]acetamide, 5-[(2-hydroxyethyl)amino]-4-methoxy-2-methylphenol, 5-[(2-hydroxyethyl)amino]-2-methylphenol, 3-[(2-hydroxyethyl)amino]phenol, 3-[(2-methoxyethyl)amino]phenol, 5-amino-2-methoxyphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 2,6-dihydroxy-3,4-dimethylpyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-dihydroxy benzene, 1-chloro-2,4-dihydroxy benzene, 2-chloro-1,3-dihydroxy benzene, 1,2-dichloro-3,5-dihydroxy-4-methylbenzene, 1,5-dichloro-2,4-dihydroxy benzene, 1,3-dihydroxy-2-methylbenzene, 3,4-methylendioxy phenol, 3,4-methylendioxy aniline, 5-[(2-hydroxyethyl)amino]-1,3-benzodioxol, 6-bromine-1-hydroxy-3,4-methylendioxy benzene, 3,4-diamino benzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione.

The above-named developer substances and coupler substances can be used in the colorant according to the present invention either singly or in mixtures with each other, wherein the total amount of developer substances and coupler substances in the agent according to the present invention is from about 0.01% to 12% by weight, or especially from about 0.2% to 6% by weight.

Moreover, the colorant according to the present invention can also contain additional color components, for example 4-(2,5-diamino benzylamino)aniline or 3-(2,5-diamino benzylamino)aniline, as well as other typical natural, natural-identical, or synthetic direct-penetrating dyes from the group of anionic (acidic) and cationic (alkaline) dyes, from triarylmethane dyes, from nitro dyes, from dispersion dyes, and from azo dyes, for example natural dyes such as indigo or henna; nitro dyes such as 1,4-bis[(2-hydroxyethyl)amino]-2-nitrobenzene, 1-(2-hydroxyethyl)amino-2-nitro-4-[di(2-hydroxyethyl)amino]benzene, (HC Blue No. 2), 1-amino-3-methyl-4-[(2-hydroxyethyl)amino]-6-nitrobenzene, (HC Violet No. 1), 4-[ethyl-(2-hydroxyethyl)amino]-1-[(2-hydroxyethyl)amino]-2-nitrobenzene hydrochloride (HC Blue No. 12), 4-[di(2-hydroxyethyl)amino]-1-[(2-methoxyethyl)amino]-2-nitrobenzene, (HC Blue No. 11), 1-[(2,3-dihydroxypropyl)amino]-4-[methyl-(2-hydroxyethyl)amino]-2-nitrobenzene (HC Blue No. 10), 1-[(2,3-dihydroxypropyl)amino]-4-[ethyl-(2-hydroxyethyl)amino]-2-nitrobenzene hydrochloride (HC Blue No. 9), 1-(3-hydroxypropylamino)-4-[di(2-hydroxyethyl)amino]-2-nitrobenzene, (HC Violet No. 2), 1-methylamino-4-[methyl-(2,3-dihydroxypropyl)amino]-2-nitrobenzene (HC Blue No. 6), 2-((4-amino-2-nitrophenyl)amino)-5-dimethylamino benzoic acid (HC Blue No. 13), 1-(2-aminoethylamino)-4-[di(2-hydroxyethyl)amino]-2-nitrobenzene, 4-(di(2-hydroxyethyl)amino)-2-nitro-1-phenylamino benzene, 1-amino-4-[(2-hydroxyethyl)amino]-2-nitrobenzene (HC Red No. 7), 2-amino-4,6-dinitrophenol, 1,4-diamino-2-nitrobenzene (CI76070), 4-amino-2-nitrodiphenylamine (HC Red No. 1), 1-amino-4-[di-(2-hydroxyethyl)amino]-2-nitrobenzene hydrochloride (HC Red No. 13), 1-amino-5-chloro-4-[(2-hydroxyethyl)amino]-2-nitrobenzene, 4-amino-1-[(2-hydroxyethyl)amino]-2-nitrobenzene (HC Red No. 3), 4-((2-hydroxyethyl)methylamino)-1-(methylamino)-2-nitrobenzene, 1-amino-4-((2,3-dihydroxypropyl)amino)-5-methyl-2-nitrobenzene, 1-amino-4-(methylamino)-2-nitrobenzene, 4-amino-2-nitro-1-((prop-2-en-1-yl)amino)benzene, 4-amino-3-nitrophenol, 4-[(2-hydroxyethyl)amino]-3-nitrophenol, 4-[(2-nitrophenyl)amino]phenol (HC Orange No. 1), 1-[(2-aminoethyl)amino]-4-(2-hydroxyethoxy)-2-nitrobenzene (HC Orange No. 2), 4-(2,3-dihydroxypropoxy)-1-[(2-hydroxyethyl)amino]-2-nitrobenzene (HC Orange No. 3), 1-amino-5-chloro-4-[(2,3-dihydroxypropyl)amino]-2-nitrobenzene (HC Red No. 10), 5-chloro-1,4-[di(2,3-dihydroxypropyl)amino]-2-nitrobenzene (HC Red No. 11), 2-[(2-hydroxyethyl)amino]-4,6-dinitro phenol, 4-ethylamino-3-nitrobenzoic acid, 2-[(4-amino-2-nitrophenyl)amino]benzoic acid, 2-chloro-6-ethylamino-4-nitrophenol, 2-amino-6-chloro-4-nitrophenol, 4-[(3-hydroxypropyl)amino]-3-nitrophenol, 2,5-diamino-6-nitropyridine, 6-amino-3-((2-hydroxyethyl)amino)-2-nitropyridine, 3-amino-6-((2-hydroxyethyl)amino)-2-nitropyridine, 3-amino-6-(ethylamino)-2-nitropyridine, 3-((2-hydroxyethyl)amino)-6-(methylamino)-2-nitropyridine, 3-amino-6-(methylamino)-2-nitropyridine, 6-(ethylamino)-3-((2-hydroxyethyl)amino)-2-nitropyridine, 1,2,3,4-tetrahydro-6-nitroquinoxaline, 7-amino-3,4-dihydro-6-nitro-2H-1,4-benzoxazine (HC Red No. 14), 1,2-diamino-4-nitrobenzene (CI76020), 1-amino-2-[(2-hydroxyethyl)amino]-5-nitrobenzene (HC Yellow No. 5), 1-(2-hydroxyethoxy)-2-[(2-hydroxyethyl)amino]-5-nitrobenzene, (HC Yellow No. 4), 1-[(2-hydroxyethyl)amino]-2-nitrobenzene (HC Yellow No. 2), 2-(di(2-hydroxyethyl)amino)-5-nitrophenol, 2-[(2-hydroxyethyl)amino]-1-methoxy-5-nitrobenzene, 2-amino-3-nitrophenol, 1-amino-2-methyl-6-nitrobenzene, 1-(2-hydroxyethoxy)-3-methylamino-4-nitrobenzene, 2,3-(dihydroxypropoxy)-3-methylamino-4-nitrobenzene, 2-[(2-hydroxyethyl)amino]-5-nitrophenol (HC Yellow No. 11), 3-[(2-aminoethyl)amino]-1-methoxy-4-nitrobenzene hydrochloride (HC Yellow No. 9), 1-[(2-ureidoethyl)amino]-4-nitrobenzene, 4-[(2,3-dihydroxypropyl)amino]-3-nitro-1-trifluoromethylbenzene (HC Yellow No. 6), 1-chloro-2,4-bis-[(2-hydroxyethyl)amino]-5-nitrobenzene (HC Yellow No. 10), 1-amino-4-((2-aminoethyl)amino)-5-methyl-2-nitrobenzene, 4-[(2-hydroxyethyl)amino]-3-nitro-1-methylbenzene, 1-chloro-4-[(2-hydroxyethyl)amino]-3-nitrobenzene (HC Yellow No. 12), 4-[(2-hydroxyethyl)amino]-3-nitro-1-trifluoromethylbenzene, (HC Yellow No. 13), 4-[(2-hydroxyethyl)amino]-3-nitro benzonitrile (HC Yellow No. 14), 4-[(2-hydroxyethyl)amino]-3-nitro benzamide (HC Yellow No. 15), 3-((2-hydroxyethyl)amino)-4-methyl-1-nitrobenzene, 4-chloro-3-((2-hydroxyethyl)amino)-1-nitrobenzene, and 2,4-dinitro-1-hydroxy-naphthalene; quinone dyes such as 1,4-di[(2,3-dihydroxypropyl)amino]-9,10-anthraquinone, 1,4-di[(2-hydroxyethyl)amino]-9,10-anthraquinone (CI61545, Disperse Blue 23), 1-[(2-hydroxyethyl)amino]-4-methylamino-9,10-anthraquinone (CI61505, Disperse Blue No. 3), 2-[(2-aminoethyl)amino]-9,10-anthraquinone (HC Orange No. 5), 1-amino-4-hydroxy-9,10-anthraquinone (CI60710, Disperse Red 15), 1-hydroxy-4-[(4-methyl-2-sulfophenyl)amino]-9,10-anthraquinone, 7-beta-D-glucopyranosyl-9,10-dihydro-1-methyl-9,10-dioxo-3,5,6,8-tetrahydroxy-2-anthracene carboxylic acid (CI75470, Natural Red 4), 1-[(3-aminopropyl)amino]-4-methylamino-9,10-anthraquinone (HC Blue No. 8), 1-[(3-aminopropyl)amino]-9,10-anthraquinone (HC Red No. 8), 1,4-diamino-2-methoxy-9,10-anthraquinone (CI62015, Disperse Red No. 11, Solvent Violet No. 26), 1,4-dihydroxy-5,8-bis[(2-hydroxyethyl)amino]-9,10-anthraquinone (CI62500, Disperse Blue No. 7, Solvent Blue No. 69), 1,4-diamino-9,10-anthraquinone (CI61100, Disperse Violet No. 1), 1-amino-4-(methylamino)-9,10-anthraquinone (CI61105, Disperse Violet No. 4, Solvent Violet No. 12), 2-hydroxy-3-methoxy-1,4-naphthoquinone, 2,5-dihydroxy-1,4-naphthoquinone, 2-hydroxy-3-methyl-1,4-naphthoquinone, N-(6-((3-chloro-4-(methylamino)phenyl)imino)-4-methyl-3-oxo-1,4-cyclohexadiene-1-yl)urea (HC Red No. 9), 2-((4-(di(2-hydroxyethyl)amino)phenyl)amino)-5-((2-hydroxyethyl)amino)-2,5-cyclohexadiene-1,4-dion (HC Green No. 1), 5-hydroxy-1,4-naphthoquinone (CI75500, Natural Brown No. 7), 2-hydroxy-1,4-naphthoquinone (CI75480, Natural Orange No. 6), 1,2-dihydro-2-(1,3-dihydro-3-oxo-2H-indol-2-yliden)-3H-indol-3-on (CI73000), and 1,3-bis(dicyanomethylene)indane; alkaline dyes such as 9-(dimethylamino)benzo[a]phenoxazin-7-ium chloride (CI51175; Basic Blue No. 6), di[4-(diethylamino)phenyl][4-(ethylamino)naphthyl]carbenium chloride (CI42595; Basic Blue No. 7), di-(4-(dimethylamino)phenyl)-(4-(methylphenylamino)naphthalen-1-yl)carbenium chloride (CI42563; Basic Blue No. 8), 3,7-di(dimethylamino)phenothiazin-5-ium chloride (CI52015; Basic Blue No. 9), di[4-(dimethylamino)phenyl][4-(phenylamino)naphthyl]carbenium chloride (CI44045; Basic Blue No. 26), 2-[(4-(ethyl(2-hydroxyethyl)amino)phenyl)azo]-6-methoxy-3-methyl benzothiazolium methylsulphate (CI11154; Basic Blue No. 41), Basic Blue No. 77, 8-amino-2-bromo-5-hydroxy-4-imino-6-[(3-(trimethylammonio)phenyl)amino]-1(4H)naphthalenone chloride (CI56059; Basic Blue No. 99), bis[4-(dimethylamino)phenyl][4-(methylamino)phenyl]carbenium chloride (CI42535; Basic Violet No. 1), tri(4-amino-3-methylphenyl)carbenium chloride (CI42520; Basic Violet No. 2), tris[4-(dimethylamino)phenyl]carbenium chloride (CI42555; Basic Violet No. 3), 2-[3,6-(diethylamino)dibenzopyranium-9-yl]benzoic acid chloride (CI45170; Basic Violet No. 10), di(4-aminophenyl)(4-amino-3-methylphenyl)carbenium chloride (CI42510; Basic Violet No. 14), 1,3-bis[(2,4-diamino-5-methylphenyl)azo]-3-methylbenzene (CI21010; Basic Brown No. 4), 1-[(4-aminophenyl)azo]-7-(trimethylammonio)-2-naphthol chloride (CI12250; Basic Brown No. 16), 3-[(4-amino-2,5-dimethoxyphenyl)azo]-N,N,N-trimethylbenzeneaminium chloride (CI112605, Basic Orange No. 69), 1-[(4-amino-2-nitrophenyl)azo]-7-(trimethylammonio)-2-naphthol chloride (Basic Brown No. 17), 1-[(4-amino-3-nitrophenyl)azo]-7-(trimethylammonio)-2-naphthol chloride (CI12251; Basic Brown No. 17), 2-((4-aminophenyl)azo)-1,3-dimethyl-1H-imidazol-3-ium chloride (Basic Orange No. 31), 3,7-diamino-2,8-dimethyl-5-phenylphenazinium chloride (CI50240; Basic Red No. 2), 1,4-dimethyl-5-[(4-(dimethylamino)phenyl)azo]-1,2,4-triazolium chloride (CI11055; Basic Red No. 22), 1,3-dimethyl-2-((4-dimethylamino)phenyl)azo-1H-imidazol-3-ium chloride (Basic Red No. 51), 2-hydroxy-1-[(2-methoxyphenyl)azo]-7-(trimethylammonio)naphthalene chloride (CI12245; Basic Red No. 76), 2-[2-((2,4-dimethoxyphenyl)amino)ethenyl]-1,3,3-trimethyl-3H-indol-1-ium chloride (CI48055; Basic Yellow No. 11), 3-methyl-1-phenyl-4-[(3-(trimethylammonio)phenyl)azo]pyrazol-5-one chloride (CI12719; Basic Yellow No. 57), di[4-(dimethylamino)phenyl]iminomethane hydrochloride (CI41000; Basic Yellow No. 2), 1-methyl-4-((methylphenylhydrazono)methyl)pyridinium methylsulphate (Basic Yellow No. 87), bis[4-(diethylamino)phenyl]phenylcarbenium bisulfate (1:1) (CI42040; Basic Green No. 1), di(4-(dimethylamino)phenyl)phenylmethanol (CI42000; Basic Green No. 4), 1-(2-morpholiniumpropylamino)-4-hydroxy-9,10-anthraquinone methylsulphate, and 1-[(3-(dimethylpropylaminium)propyl)amino]-4-(methylamino)-9,10-anthraquinone chloride; neutral dyes such as 1-[di(2-hydroxyethyl)amino]-3-methyl-4-[(4-nitrophenyl)azo]benzene (CI11210, Disperse Red No. 17), 1-[di(2-hydroxyethyl)amino]-4-[(4-nitrophenyl)azo]benzene, (Disperse Black No. 9), 4-[(4-aminophenyl)azo]-1-[di(2-hydroxyethyl)amino]-3-methylbenzene, (HC Yellow No. 7), 2,6-diamino-3-[(pyridin-3-yl)azo]pyridine, 2-((4-(acetylamino)phenyl)azo)-4-methylphenol (CI11855; Disperse Yellow No. 3), and 2-((4-(ethyl(2-hydroxyethyl)amino)-2-methylphenyl)azo)-5-nitro-1,3-thiazole (CI11935; Disperse Blue No. 106); acidic dyes such as 6-hydroxy-5-[(4-sulfophenyl)azo]-2-naphthalinsulfonic acid disodium salt (CI15985; Food Yellow No. 3; FD&C Yellow No. 6), 2,4-dinitro-1-naphthol-7-sulfonic acid disodium salt (CI10316; Acid Yellow No. 1; Food Yellow No. 1), 2-(indan-1,3-dion-2-yl)quinoline-x,x-sulfonic acid (mixture of mono- and disulfonic acid) (CI47005; D&C Yellow No. 10; Food Yellow No. 13, Acid Yellow No. 3), 5-hydroxy-1-(4-sulfophenyl)-4-[(4-sulfophenyl)azo]pyrazol-3-carboxylic acid trisodium salt (CI19140; Food Yellow No. 4; Acid Yellow No. 23), 9-(2-carboxyphenyl)-6-hydroxy-3H-xanthen-3-one (CI45350; Acid Yellow No. 73; D&C Yellow No. 8), 4-((4-amino-3-sulfophenyl)azo)benzene sulfonic acid disodium salt (CI13015, Acid Yellow No. 9), 5-[(2,4-dinitrophenyl)amino]-2-phenylaminobenzenesulfonic acid sodium salt (CI10385; Acid Orange No. 3), 4-[(2,4-dihydroxyphenyl)azo]benzenesulfonic acid monosodium salt (CI14270; Acid Orange No. 6), 4-[(2-hydroxynaphth-1-yl)azo]benzenesulfonic acid sodium salt (CI15510; Acid Orange No. 7), 4-((2-hydroxy-naphthalin-1-yl)azo)-3-methylbenzene sulfonic acid sodium salt (CI15575; Acid Orange No. 8), 4-[(2,4-dihydroxy-3-[(2,4-dimethylphenyl)azo]phenyl)azo]benzene sulfonic acid sodium salt (CI20170; Acid Orange No. 24), 3′,6′-dihydroxy-4′,5′-diiodospiro(isobenzofuran-1(3H)-9′-(9H)xanthene)-3-on (CI45425, D&C Orange No. 10), 4-hydroxy-3-[(4-sulfonaphth-1-yl)azo]-1-naphthalinsulfonic acid disodium salt (CI14720; Acid Red No. 14), 4-hydroxy-3-[(2-methoxyphenyl)azo]-1-naphthalinsulfonic acid monosodium salt (CI14710; Acid Red No. 4), 6-hydroxy-5-[(4-sulfonaphth-1-yl)azo]-2,4-naphthalenedisulfonic acid trisodium salt (CI16255; Ponceau 4R; Acid Red No. 18), 3-hydroxy-4-[(4-sulfonaphth-1-yl)azo]-2,7-naphthalene disulfonic acid trisodium salt (CI16185; Acid Red No. 27), 8-amino-1-hydroxy-2-(phenylazo)-3,6-naphthalenedisulfonic acid disodium salt (CI17200; Acid Red No. 33), 5-(acetylamino)-4-hydroxy-3-[(2-methylphenyl)azo]-2,7-naphthalenedisulfonic acid disodium salt (CI18065; Acid Red No. 35), 2-(3-hydroxy-2,4,5,7-tetraiod dibenzopyran-6-on-9-yl)benzoic acid disodium salt (CI45430; Acid Red No. 51), N-[6-(diethylamino)-9-(2,4-disulfophenyl)-3H-xanthen-3-ylidene]-N-ethylethanammonium hydroxide, inner salt, sodium salt (CI45100; Acid Red No. 52), 8-[(4-(phenylazo)phenyl)azo]-7-naphthol-1,3-disulfonic acid disodium salt (CI27290; Acid Red No. 73), 2′,4′,5′,7′-tetrabromo-3′,6′-dihydroxyspiro[isobenzofuran-1(3H),9′-[9H]xanthene]-3-on-dinatriumsalz (CI45380; Acid Red No. 87), 2′,4′,5′,7′-tetrabromo-4,5,6,7-tetrachloro-3′,6′-dihydroxyspiro[isobenzofuran-1(3H),9′[9H]xanthen]-3-one disodium salt (CI45410; Acid Red No. 92), 3′,6′-dihydroxy-4′,5′-diiodospiro[isobenzofuran-1(3H),9′(9H)-xanthene]-3-on-disodium salt (CI45425; Acid Red No. 95), 2-hydroxy-3-((2-hydroxynaphth-1-yl)azo)-5-nitrobenzene sulfonic acid monosodium salt (CI15685; Acid Red No. 184), (2-sulfophenyl)di[4-(ethyl((4-sulfophenyl)methyl)amino)phenyl]carbenium disodium salt, betaine (CI42090; Acid Blue No. 9; FD&C Blue No. 1), 3-hydroxy-4-((4-methyl-2-sulfophenyl)azo)-2-naphthalene carboxylic acid disodium salt (CI15850; D&C Red No. 6), 6-hydroxy-5-((2-methoxy-5-methyl-4-sulfophenyl)azo)-2-naphthalinsulfonic acid disodium salt (CI16035; FD&C Red 40), 1,4-bis[(2-sulfo-4-methylphenyl)amino]-9,10-anthraquinone disodium salt (CI61570; Acid Green No. 25), bis-[4-(dimethylamino)phenyl]-(3,7-disulfo-2-hydroxynaphth-1-yl)carbenium inner salt, monosodium salt (CI44090; Food Green No. 4; Acid Green No. 50), bis-[4-(diethylamino)phenyl](2,4-disulfophenyl)carbenium inner salt, sodium salt (2:1) (CI42045; Food Blue No. 3; Acid Blue No. 1), bis-[4-(diethylamino)phenyl](5-hydroxy-2,4-disulfophenyl)carbenium inner salt, calcium salt (2:1) (CI42051; Acid Blue No. 3), 1-amino-4-(cyclohexylamino)-9,10-anthraquinone-2-sulfonic acid sodium salt (CI62045; Acid Blue No. 62), 3,3-bis(3,5-dibromo-4-hydroxyphenyl)-4,5,6,7-tetrabromo-2,1(3h)-benzoxathiol-1,1-dioxide, 1-amino-4-(phenylamino)-9,10-anthraquinone-2-sulfonic acid (CI62055; Acid Blue No. 25), 2-(1,3-dihydro-3-oxo-5-sulfo-2H-indol-2-yliden)-2,3-dihydro-3-oxo-1H-indol-5-sulfonic acid disodium salt (CI73015; Acid Blue No. 74), 9-(2-carboxyphenyl)-3-[(2-methylphenyl)amino]-6-[(2-methyl-4-sulfophenyl)amino]xanthylium inner salt, monosodium salt (CI45190; Acid Violet No. 9), 1-hydroxy-4-[(4-methyl-2-sulfophenyl)amino]-9,10-anthraquinone sodium salt (CI60730; D&C Violet No. 2; Acid Violet No. 43), bis-[3-nitro-4-[(4-phenylamino)-3-sulfophenylamino]phenyl] sulfone (CI10410; Acid Brown No. 13), 5-amino-4-hydroxy-6-[(4-nitrophenyl)azo]-3-(phenylazo)-2,7-naphthalenedisulfonic acid disodium salt (CI20470; Acid Black No. 1), 3-hydroxy-4-[(2-hydroxynaphth-1-yl)azo]-7-nitro-1-naphthalenesulfonic acid chromium complex (3:2) (CI15711; Acid Black No. 52), 3-[(2,4-dimethyl-5-sulfophenyl)azo]-4-hydroxy-1-naphthalenesulfonic acid disodium salt (CI14700; Food Red No. 1; Ponceau SX; FD&C Red No. 4), 4-(acetylamino)-5-hydroxy-6-[(7-sulfo-4-[(4-sulfophenyl)azo]naphth-1-yl)azo]-1,7-naphthalene disulfonic acid tetrasodium salt (CI28440; Food Black No. 1), and 3-hydroxy-4-(3-methyl-5-oxo-1-phenyl-4,5-dihydro-1H-pyrazol-4-ylazo)naphthalene-1-sulfonic acid sodium salt, chromium complex (Acid Red No. 195).

Further dyes that are conventional and known for use in hair coloring, among others, are described in E. Sagarin, Cosmetics, Science and Technology, Interscience Publishers Inc., New York (1957), p. 503 ff.; as well as Janistyn, Handbuch der Kosmetika und Riechstoffe [Handbook of Cosmetics and Scents], vol. 3 (1973), p. 388 ff.; and K. Schrader Grundlagen und Rezepturen der Kosmetika [Fundamentals and Formulations in Cosmetics], 2nd edition (1989), pp. 782-815, to which express reference is made.

The colorant can contain the other aforementioned color components in a total quantity of about 0.001% to 6% by weight.

The aforementioned developer substances and/or coupler substances and/or other color components enable a multitude of different color shades in conjunction with the combination according to the invention of 2-amino-5-ethylphenol and m-aminophenoles of general Formula (I). Thus, for example, it is possible to obtain brilliant blonde to brown hair colors by using a combination of 2-amino-5-ethylphenol and m-aminophenoles of general Formula (I) as well as 4-(2,5-diamino benzylamino)aniline and/or 2-(3-aminophenyl)aminomethyl-1,4-diaminobenzene, or salts thereof.

Of course, additional coupler substances as well as developer substances and other color components can be used, whereas if they are bases, they can also be used in the form of their physiologically compatible salts with organic or inorganic acids such as, for example, hydrochloric acid or sulfuric acid, while if they possess aromatic OH-groups, they can also be used in the form of their salts with bases, for example, as alkali phenolates.

Moreover, if these colorants are to be used in the coloring of hair, they can contain further conventional cosmetic additives, for example antioxidants such as ascorbic acid, thioglycolic acid, or sodium sulfite, as well as perfume oils, chelating agents, wetting agents, emulsifiers, thickeners, and conditioning agents.

The formulation of the colorant according to the present invention can be, for example, a solution, in particular an aqueous or aqueous alcoholic solution, a paste, a cream, a powder, a granulate, a gel, an emulsion, a dispersion, or an aerosol preparation. The composition of the solution represents a mixture of dye components with the additives conventional for such formulations.

Conventional additives in solutions, creams, emulsions, dispersions, or gels are, for example, solvents such as water, lower aliphatic alcohols for example ethanol, propanol, or isopropanol, glycerin or glycols such as 1,2-propyleneglycol, and, furthermore, wetting agents or emulsifiers from the anionic, cationic, amphoteric, or nonionic classes of surface-active substances such as, for example, fatty alcohol sulfates, oxyethylated fatty alcohol sulfates, alkyl sulfonates, alkylbenzene sulfonates, alkyltrimethylammonium salts, alkyl betaines, oxyethylated fatty alcohols, oxyethylated nonylphenols, fatty acid alkanolamides, and oxyethylated fatty acid esters, and, moreover, thickeners such as higher fatty alcohols, starches, cellulose derivatives, petroleum jelly, paraffin oil, and fatty acids, and conditioning agents such as cationic resins, lanolin derivatives, cholesterol, pantothenic acid, and betaine. The aforementioned components are employed in the amounts usual for such purposes, for example, wetting agents and emulsifiers in concentrations of from about 0.5% to 30% by weight, thickeners in amounts of from about 0.1% to 30% by weight, and conditioning agents in concentrations of from about 0.1% to 5% by weight.

Suitable carrier materials for the powder or granulate applications are powdery, micro-crystalline substances that displace the dyes in a physical condition that enables the method for coating the pellets to be conducted with suitable encapsulating materials. Suitable carrier materials are, in particular, arabica gum, dextrose, polyvinylpyrrolidone, oligosaccharides, micro-crystalline cellulose derivatives such as hydroxypropyl methylcellulose, methylcellulose, hydroxyethyl cellulose, hydroxypropyl cellulose, nonoxynol hydroxyethyl cellulose, and cetyl hydroxyethyl cellulose, or physically or chemically modified starches or starch derivatives such as starch esters (for example acetylated starches), starch ethers (for example hydroxyalkylated starches), dialdehyde starches, dicarboxyl starches, distarch phosphates, hydroxyalkyl starch phosphates, or hydroxyalkyl starches, wherein the alkyl groups preferably contain 1 to 4, or particularly preferably 2 to 3, C atoms. Cross-linked starch ethers such as those with the INCI designations Dimethylimidazolidone Rice or Corn Starch or hydrophobically modified starches (for example those with the INCI designation Aluminium Starch Octensuccinate) are suitable. The starches can have been modified thermally, hydrolytically, or enzymatically, wherein the starting starches can be recovered from known sources, for example corn, potatoes, sweet potatoes, peas, bananas, oats, wheat, barley, rice, sago, tapioca, arrowroot, amaranth, canna, sorghum, etc. Particularly preferable starch derivatives are nonionic starch derivatives, particularly with alkylene oxides such as ethylene oxide, propylene oxide, or butylene oxide, acetane hydride, or butyl keten dimer, and particularly propylene oxide, and modified nonionic starch derivatives. Other suitable carrier materials are synthetic calcium silicate, diatomaceous earth, silicium dioxide, or other free-flowing non-caking powders.

Suitable encapsulating materials for the powders or granulates are soluble or water-dispersible, film-forming substances that are capable, from solutions or dispersions, of depositing uniform films on the powders via spray drying, which can be referred to as coating.

Suitable encapsulating materials are arabica gum, cellulose derivatives (for example methylcelluloses), polyethylene dispersions, polyacrylic acids, polyvinyl alcohols, polycarbonates, polyvinylpyrrolidone, polyesters, and polyamides, or natural film-formers such as chitosan, shellac, oligosaccharides, or also Chinese balsam resin (colophonium).

The powdery compositions can also be de-dusted by an existing, hydrophobic base of oils and waxes, for example of an organically lipophilic compound from the group of vegetable and/or animal fats, oils, and waxes, of paraffin hydrocarbons, of higher alcohols and ethers, of aliphatic and aromatic esters, as well as of silicon oils.

Likewise, it is possible to form suspensions, which can also contain at least one inorganic or organic thickener with a lipophilic character, which forms an oleo gel or lipo gel with the lipophilic compound.

Vegetable oils such as jojoba oil; Vaseline; liquid paraffins, for example paraffinum perliquidum and paraffinum subliquidum; silicone oils; liquid, long-chain, hydrophobic fatty acid esters for example octyl palmitate, isocetyl palmitate, isopropyl palmitate, and octyl stearate; waxy long-chain, hydrophobic fatty acid esters and/or synthetic wax replacement materials for example natural or synthetic beeswax (for example Lipowachs 6138G® sold by Lipo Chemicals), C18 to C36 fatty acids (for example Synchrowachs AW1C® sold by Croda Chemicals Ltd.), C10 to C36 fatty acid triglycerides such as octanoic acid/lauric acid triglyceride, hydrogenated coco fatty acid glycerides (for example Softisan 100® sold by Hüls AG), glyceryl tribehenate (for example Synchrowachs HRC® sold by Croda Chemicals Ltd.), fatty acid ester mixtures (for example Cutina BW® sold by Henkel KGaA), as well as mixtures thereof are particularly suitable organic lipophilic compounds.

The use of jojoba oil, fatty acid esters, paraffin oil, combinations of fatty acid esters and paraffin oil, as well as combinations of fatty acid esters and/or paraffin oil with Vaseline is especially preferred.

The lipophilic compounds are used in a total quantity of about 0.1% to 80% by weight, with from 3% to 65% by weight being preferred, and from 20% to 50% by weight being particularly preferred, in relation to the total quantity of bleaching agent suspension.

Alkali carboxylates, alkaline earth carboxylates, or aluminum carboxylates, for example sodium palmitate, aluminum/magnesium hydroxy stearate, or magnesium stearate, aluminum monostearate, aluminum monodistearate, and/or aluminum tristearate; copolymerisates of alkenes such as ethylene/propylene copolymers; crosslinked organic polymers and lypophilized layer silicates such as benzyl dimethyl stearyl ammonium hectorite (for example Bentone 28 sold by Rheox), as well mixtures of these lipophilic thickeners can be mentioned as lipophilic inorganic or organic thickeners, wherein alkali stearates, alkaline earth stearates, aluminum stearates, and aluminum/magnesium hydroxy stearate, and particularly magnesium stearates and aluminum stearates are preferred.

Depending on the composition, the dye according to the present invention can be weakly acidic, neutral, or alkaline.

The colorant according to the invention can be used either with an oxidizing agent or with “air oxidation,” wherein, if necessary, a suitable enzyme system is added.

For use with oxidizing agents, the above-described colorant is mixed with an oxidizing agent immediately before use, and an amount of this mixture that is sufficient for a hair coloring treatment, which is about 60 g to 200 g depending on the relative thickness of the hair, is applied to the hair.

The agent according to the invention can contain at least one source of an oxidizing agent. Preferred oxidizing agents are water-soluble peroxide-containing oxidizing agents. “Water-soluble” in this case means that, under standard conditions, at least 0.1 g, or preferably 1 g, and particularly 10 g of the oxidizing agent can be dissolved in 1 liter of demineralized water. The oxidizing agents are useable for the initial solubilization and decolorization of the melanin (bleaching) and the oxidation of the oxidative dye precursors (oxidative coloring) in the shaft of the hair.

Any known water-soluble oxidizing agent can be used within the scope of the present invention, wherein inorganic peroxides that form hydrogen peroxide in an aqueous solution are preferred. Water-soluble peroxide-containing oxidizing agents are well known from the prior art and contain hydrogen peroxide, inorganic metallic peroxides such as sodium periodate or sodium peroxide, organic peroxides such as urea peroxide or melamine peroxide, and inorganic persalt bleaching agents such as alkali metallic salts of perborates, percarbonates, perphosphates, persilicates, persulfates, and the like. These inorganic persalts can be contained as monohydrates, tetrahydrates, etc. Alkyl and aryl peroxides and/or peroxidases can also be used. If desired, mixtures of 2 or more of these oxidizing agents can be used. The oxidizing agents can be present in the form of an aqueous solution or a powder, which is dissolved before application. Preferred oxidizing agents according to the present invention are hydrogen peroxide, percarbonates, persulfates, and combinations of these compounds.

According to the present invention, the agents contain about 0.1% to about 15% by weight, preferably about 1% to 10% by weight, and particularly about 2% to about 7% by weight of the oxidizing agent.

An additional preferred oxidizing agent is a source of peroxy mono carbonation, which is preferably formed in situ from a hydrogen peroxide source and a bicarbonate source. These types of oxidizing agents are particularly effective at a pH value that is less than or equal to 9.5, wherein a pH value of 7.5 to 9.5 and particularly about 9 is preferred. Furthermore, this system is particularly effective in conjunction with a source of ammonia or ammonium ions. It has been shown that oxidizing agents can positively influence the desired hair coloring results while simultaneously significantly reducing the smell, the skin and scalp irritations, as well as damage to the hair.

Any source of these ions can be used, wherein for example sodium, potassium, guanidine, arginine, lithium, calcium, magnesium, barium, or ammonium salts of carbonates, carbamates, and bicarbonates, as well as mixtures thereof, for example sodium carbonate, sodium bicarbonate, potassium carbonate, potassium hydrogen carbonate, guanidine carbonate, guanidine hydrogen carbonate, lithium carbonate, calcium carbonate, magnesium carbonate, barium carbonate, ammonium carbonate, ammonium hydrogen carbonate, and mixtures thereof can be mentioned as suitable sources. Percarbonate salts can be used as both a carbonation source and an oxidizing agent. Preferred sources of carbonates, carbamates, and bicarbonates are sodium bicarbonate, potassium hydrogen carbonate, and ammonium carbamate, as well as mixtures thereof.

According to the present invention, the agents contain about 0.1% to about 15% by weight, or preferably about 1% to about 10% by weight, and particularly about 1% to about 8% by weight of a hydrogen carbonation source and about 0.1% to 10% by weight, or preferably about 1% to 7% by weight, and particularly about 2% to about 6% by weight of a hydrogen peroxide source.

According to the present invention, the agents, if necessary, can also contain at least one source of an alkalizing agent, preferably a source of ammonia or ammonium ions.

Any known compound, for example alkanolamides such as monoethanolamine, diethanolamine, triethanolamine, monopropanolamine, dipropanolamine, tripropanolamine, 2-amino-2-methyl-1,3-propandiol, 2-amino-2-methyl-1-propanol, or 2-amino-2-hydroxymethyl-1,3-propandiol, and guanidium salts, can be used as an alkalizing agent. Particularly preferred alkalizing agents are those agents having a source of ammonium ions, wherein any source of ammonium ions is suitable. Preferred sources of ammonium ions are ammonium chloride, ammonium sulfate, ammonium nitrate, ammonium phosphate, ammonium acetate, ammonium carbonate, ammonium hydrogen carbonate, ammonium carbamate, ammonium hydroxide, percarbonate salts, ammonium, and mixtures thereof. Ammonium carbonate, ammonium carbamate, ammonium hydrogen carbonate, ammonia, and mixtures thereof are particularly preferred in this case. The agents according to the present invention can contain about 0.1% to about 10% by weight, or preferably about 0.5% to about 5% by weight, and particularly about 1% to about 3% by weight of an alkalizing agent, preferably ammonium ions.

According to the present invention, the agents can also contain a source of a radical scavenger. The term “radical scavenger” in the case at hand describes a compound that reacts with a reactive radical, preferably carbonate radicals, in order to convert these reactive radicals into a less reactive compound using a series of quick reactions.

Suitable radical scavengers are compounds of general Formula (I)
R¹—Y—C(H)(R³)—R⁴—(C(H)(R⁵)—Y—R⁶)ₙ (I)
with Y equal to NR², O, or S, preferably NR², n equal to 0 to 2, and wherein R⁴ is a monovalent or divalent radical from the group consisting of: (a) substituted or unsubstituted, straight-chain or branched alkyl or monounsaturated or polyunsaturated alkyl or heteroalkyl groups and aliphatic, heteroaliphatic, or heteroolefinic systems; (b) substituted or unsubstituted, mono- or polycyclic aliphatic, aromatic, or heterocyclic systems; or (c) substituted or unsubstituted, mono-, poly- or perfluorated alkyl systems; wherein systems (a), (b), and (c) have 1 to 12 carbon atoms and 0 to 5 heteroatoms (O, S, N, P, Si); and wherein R⁴ together with R³ or R⁵ form a 5-, 6-, or 7-member ring; and wherein R¹, R², R³, R⁵, and R⁶ are monovalent, and, independently from one another, represent one of aforementioned groups (a), (b), or (c), or hydrogen.

R⁴ is preferably equal to (a) a substituted or unsubstituted, straight-chain or branched alkyl or heteroalkyl group, or an aliphatic, heteroaliphatic, or heteroolefinic system; (b) a substituted or unsubstituted, mono- or polycyclic aliphatic, aromatic, or heterocyclic system; or (c) a substituted or unsubstituted, mono-, poly-, or perfluorated alkyl system, wherein R⁴ being equal to (a) a substituted or unsubstituted, straight-chain or branched alkyl or heteroalkyl group, or an aliphatic or heteroaliphatic system; (b) a substituted or unsubstituted aromatic or heterocyclic system; (c) a substituted or unsubstituted, mono-, poly-, or perfluorated alkyl system, and particularly a substituted or unsubstituted, straight-chain or branched alkyl or heteroalkyl group is especially preferred.

Preferably, the previously described R⁴ groups (a), (b), and (c) have 1 to 8 carbon atoms, wherein 1 to 6 carbon atoms and particularly 1 to 4 carbon atoms are particularly preferred, as well as 0 to 3 heteroatoms, preferably 0 to 2 heteroatoms, and particularly 1 heteroatom. If the system has heteroatoms, it preferably contains 1 heteroatom. The following can be mentioned in particular as a heteroatom: O, S, and N, wherein O and N, and particularly O are preferred.

R¹, R², R³, R⁵, and R⁶, independently from one another, represent hydrogen or one of the systems mentioned for R^{4.}

Alternatively, R¹, R², R³, R⁴, R⁵, and R⁶ can also be substituted, wherein the substituent is preferably selected from: (a) C-linked monovalent substituents from the group of (i) substituted or unsubstituted, straight-chain or branched alkyl groups, monounsaturated or polyunsaturated alkyl, heteroalkyl, aliphatic, heteroaliphatic, or heteroolefinic systems; (ii) substituted or unsubstituted, mono- or polycyclic aliphatic, aromatic or heterocyclic systems; or (iii) substituted or unsubstituted, mono-, poly-, or perfluoroalkyl systems; wherein aforementioned systems (i), (ii), and (iii) contain 1 to 10 carbon atoms and 0 to 5 heteroatoms (O, S, N, P, Si); (b) S-linked monovalent substituents from the group SA¹, SCN, SO₂A¹, SO₃A¹, SSA¹, SOA¹, SO₂NA¹A², SNA¹A², and SONA¹A²; (c) O-linked monovalent substituents from the group OA¹, OCN, and ONA¹A²; (d) N-linked monovalent substituents from the group NA¹A², (NA¹A²A³)⁺, NC, NA¹OA², NA¹SA², NCO, NCS, NO₂, N=NA¹, N=NOA¹, NA¹CN, NA¹NA²A³; (e) monovalent substituents COOA¹, CON₃, CONA¹₂, CONA¹COA², C(=NA¹)NA¹A², CHO, CHS, CN, NC, and X; and (f) fluoroalkyl monovalent substituents from the group of mono-, poly-, or perfluoroalkyl systems with 1 to 12 carbon atoms and 0 to 4 heteroatoms.

In the aforementioned groups (b) through (e), A¹, A², and A³ are monovalent and, independently from one another, represent (1) H, (2) substituted or unsubstituted, straight-chain or branched alkyl groups, monounsaturated or polyunsaturated alkyl groups, heteroalkyl groups, aliphatic, heteroaliphatic, or heteroolefinic systems, (3) substituted or unsubstituted, mono- or polycyclic aliphatic, aromatic or heterocylic systems, or (4) substituted or unsubstituted, mono-, poly-, or perfluoroalkyl systems; wherein aforementioned systems (2), (3), and (4) have 1 to 10 carbon atoms and 0 to 5 heteroatoms (O, S, N, P, Si); and wherein X is a halogen atom (F, Cl, Br, I).

Preferred substituents have a Hammett sigma para (óₚ) value of −0.65 to +0.75, or preferably −0.4 to +0.5. Hammett sigma values are described in the literature in Advanced Organic Chemistry—Reactions, Mechanisms and Structure (Jerry March, 5^{th} ed. (2001) pp. 368-375), to which express reference is made.

Likewise suitable radical scavengers are compounds of general Formula (II) 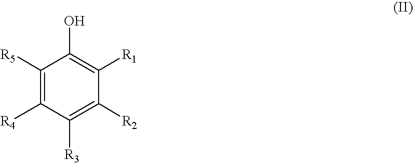
with R₁, R₂, R₃, R₄, and R₅, independently from one another, equaling H, COO⁻M⁺, Cl, Br, SO₃⁻M⁺, NO₂, OCH₃, OH or a C¹ to C¹⁰ primary or secondary alkyl group, and M equals either H or an alkali metal atom. Preferably, the aforementioned radical scavengers have a pKa value of 8.5 to ensure protonation of the hydroxyl group.

Other suitable radical scavengers are those selected from group (III) consisting of benzylamine, imidazole, di-tert-butyl hydroxy toluene, hydroquinone, guanine, pyrazine, piperidine, morpholine, methylmorpholine, 2-methoxyethylamine, and mixtures thereof.

According to the present invention, preferred radical scavengers are alkanolamines, amino sugars, amino acids, amino acid ester, and mixtures thereof. The following are especially preferred: monoethanolamine, 3-amino-1-propanol, 4-amino-1-butanol, 5-amino-1-pentanol, 1-amino-2-propanol, 1-amino-2-butanol, 1-amino-2-pentanol, 1-amino-3-pentanol, 1-amino-4-pentanol, 3-amino-2-methylpropan-1-ol, 1-amino-2-methylpropan-2-ol, 3-aminopropan-1,2-diol, glucosamine, N-acetylglucosamine, glycine, arginine, lysine, proline, glutamine, histidine, sarcosine, serine, glutamine acid, tryptophan, and mixtures thereof, as well as salts thereof such as potassium, sodium, and ammonium salts or mixtures thereof. Especially preferred compounds are glycine, sarcosine, lysine, serine, 2-methoxyethylamine, glucosamine, glutamine acid, morpholine, piperdine, ethylamine, 3-amino-1-propanol, and mixtures thereof.

The radical scavengers according to the present invention preferably have a molecular weight less than about 500, or preferably less than about 300, or particularly less than about 250, to facilitate penetration of the radical scavenger into the hair fiber. The agents according to the present invention contain, if they contain a radical scavenger, preferably about 0.1% to about 10% by weight, or particularly 1% to 7% by weight, of the radical scavenger. The radical scavenger is preferably selected so that it is not the same type of compound as that of the alkalizing agent. According to one embodiment of the present invention, the radical scavenger can also be formed in situ in the hair colorant before application.

Hydrogen peroxide or its addition compounds with urea, melamine, sodium borate, or sodium carbonate in the form of a from 3% to 12%, preferably 6%, aqueous solution, and also oxygen in the air can all be given primary consideration as suitable oxidizing agent for developing the hair coloring. If a 6% hydrogen peroxide solution is used as the oxidizing agent, the weight ratio between the hair dye and the oxidizing agent will be from 5:1 to 1:3; however it is preferably 1:1 to 1:2. In particular, larger amounts of the oxidizing agent are used with higher dye concentrations in the hair dye, or if a stronger bleaching of the hair is desired at the same time.

The pH value of the ready-to-use colorant is preferably equal to 4 to 11 and is set when the colorant is mixed with the oxidizing agent. If necessary, the pH value can be set by adding suitable acids or bases.

The mixture is allowed to react with the hair at from 15 to 50° C. for a period of about 5 to 60 minutes, and preferably for a period of 10 to 45 minutes, after which the hair is rinsed with water and dried. If necessary, the hair is subsequently washed with shampoo and re-rinsed, as needed, with a weak organic acid such as citric acid or tartaric acid. Next the hair is dried.

The colorant according to the invention containing 2-amino-5-ethylphenol and at least one m-aminophenol of general Formula (I) enables an excellent color balance from the hair roots to the tips and reduces, in an excellent manner, the effect of “matte formation” on the previously colored but still healthy hair.

The hair dye according to the invention, depending on the type and composition of the color components, enables a broad palette of different color shades, which cover the range from blonde through brown, yellow-orange, crimson, and violet all the way to blue and black color tones.

The following examples will illustrate the object of the invention without limiting the invention to these examples.

### EXAMPLES

### Examples 1 through 45

### Hair Dye

W g 2-amino-5-ethylphenol

X g 3-amino-2,6-dimethylphenol

Y g 5-((2-hydroxyethyl)amino)-2-methylphenol

Z g 5-amino-2-ethylphenol

T g developer substance E8 through E15 according to Table 1

U g coupler substance K11 through K30 according to Table 3

V g direct-penetrating dye D3 and D4 according to Table 2

10.0 g potassium oleate (8% aqueous solution)

10.0 g ammonia (22% aqueous solution)

10.0 g ethanol
0.3 g ascorbic acid balance to

100.0 g water

30 g of the present dye solution is mixed with 30 g of a 6% aqueous hydrogen peroxide solution immediately before use. Then the mixture is applied to bleached hair. After an action period of 30 minutes at 40° C. the hair is rinsed with water, washed with a commercially available shampoo, and dried. The coloring results are compiled in Table 5.

### Examples 46 through 64

### Hair Colorant

| W | g | 2-amino-5-ethylphenol |
|---|---|---|
| X | g | 3-amino-2,6-dimethylphenol |
| Y | g | 5-((2-hydroxyethyl)amino)-2-methylphenol |
| Z | g | 5-amino-2-ethylphenol |
| T | g | developer substance E8 through E15 according to Table 1 |
| U | g | coupler substance K12 through K42 according to Table 3 |
| V | g | direct-penetrating dye D1 to D9 according to Table 2 |
| 15.0 | g | cetyl alcohol |
| 0.3 | g | ascorbic acid |
| 3.5 | g | sodium lauryl alcohol diglycol ether sulfate, |
| | | 28% aqueous solution |
| 3.0 | g | ammonia 22% aqueous solution |
| 0.3 | g | sodium sulfite, anhydrous |

| | |
|---|---|
| balance to | water |
| 100.0 g | |

30 g of the present coloring cream is mixed with 30 g of a 6% hydrogen peroxide solution immediately before use. Then the mixture is applied to the hair. After an action period of 30 minutes, the hair is rinsed with water, washed with a commercially available shampoo, and dried. The coloring results are compiled in Table 6.

### Examples 65 to 72

### Hair Colorant

Hair coloring solutions of the following composition were prepared:

| W | g | 2-amino-5-ethylphenol |
|---|---|---|
| X | g | 3-amino-2,6-dimethylphenol |
| Y | g | 5-((2-hydroxyethyl)amino)-2-methylphenol |
| Z | g | 5-amino-2-ethylphenol |
| T | g | developer substance E8 through E15 according to Table 1 |
| U | g | color components FK1 and/or FK2 according to Table 4 |
| 10.0 | g | potassium oleate (8% aqueous solution) |
| 10.0 | g | ammonia (22% aqueous solution) |
| 10.0 | g | ethanol |
| 0.3 | g | ascorbic acid |

| | |
|---|---|
| balance to | water |
| 100.0 g | |

30 g of the present coloring solution is mixed with 30 g of a 6% hydrogen peroxide solution immediately before use. Then the mixture is applied to bleached hair. After an action period of 30 minutes at 40° C. the hair is rinsed with water, washed with a commercially available shampoo, and dried. The coloring results are compiled in Table 7.

TABLE 1
Developer substances

| | |
|---|---|
| E8 | 1,4-diaminobenzene |
| E9 | 2,5-diaminophenylethanol sulfate |
| E10 | 3-methyl-4-aminophenol |
| E11 | 4-amino-2-aminomethylphenol dihydrochloride |
| E12 | 4-aminophenol |
| E13 | N,N-bis(2′-hdroxyethyl)-p-phenylenediamine sulfate |
| E14 | 4,5-diamino-1-(2′-hydroxyethyl)pyrazole sulfate |
| E15 | 2,5-diaminotoluene sulfate |

TABLE 2
Direct-penetrating dyes

| | |
|---|---|
| D1 | 2,6-diamino-3-((pyridin-3-yl)azo)pyridine |
| D2 | 6-chloro-2-ethylamino-4-nitrophenol |
| D3 | 2-amino-6-chloro-4-nitrophenol |
| D4 | hydroxyethyl-2-nitro-p-toluidine |
| D5 | 4-nitrophenyl aminoethyl urea |
| D6 | N-(2-hydroxyethyl)-2-nitro-4-(trifluoromethyl)aniline |
| D7 | 2-hydroxyethyl picramic acid |
| D8 | 3-(4-amino-2-chloro-5-nitrophenyl)amino-1,2-propandiol |
| D9 | 3-nitro-p-hydroxyethyl aminophenol |

TABLE 3
Coupler substances

| | |
|---|---|
| K11 | 1,3-diaminobenzene |
| K12 | 2-amino-4-(2′-hydroxyethyl)aminoanisole sulfate |
| K13 | 1,3-diamino-4-(2′-hydroxyethoxy)benzene sulfate |
| K14 | 2,4-diamino-5-fluorotoluene sulfate |
| K15 | 3-amino-2-methylamino-6-methoxy pyridine |
| K16 | 3,5-diamino-2,6-dimethoxypyridine dihydrochloride |
| K17 | 2,4-diamino-5-ethoxytoluene sulfate |
| K18 | N-(3-dimethylamino)phenyl urea |
| K19 | 1,3-bis(2,4-diaminophenoxy)propane tetrahydrochloride |
| K20 | 3-aminophenol |
| K21 | 5-amino-2-methylphenol |
| K22 | 3-amino-2-chloro-6-methylphenol |
| K23 | 5-amino-4-fluoro-2-methylphenol sulfate |
| K24 | 1-naphthol |
| K26 | 1,3-dihydroxybenzene |
| K27 | 2-methyl-1,3-dihydroxybenzene |
| K28 | 1-chloro-2,4-dihydroxybenzene |
| K29 | 4-(2′-hydroxyethyl)amino-1,2-methylendioxybenzene*HCl |
| K30 | 3,4-methylenedioxyphenol |
| K31 | 1,5-naphthalenediol |
| K32 | 2,7-naphthalenediol |
| K33 | 1,2,4-trihydroxybenzene |
| K34 | phenyl methylpyrazole |
| K35 | 6-amino-o-cresol |
| K36 | 2,6-dihydroxy-3,4-dimethylpyridine |
| K37 | 2-amino-3-hydroxypyridine |
| K38 | 4,6-diamino-5-methoxy-2-(methylamino)-1H-benzimidazole |
| K39 | 2-amino-5-[(2-pyridinylamino)methyl]phenol |
| K40 | 2-methyl-5-[(3-pyridinylmethylene)amino]phenol |
| K41 | 4-{[(3-hydroxy-4-methylphenyl)imino]methyl}-2-methoxyphenol |
| K42 | 1,3-dihydroxy-2,4-dimethylbenzene |

TABLE 4
Color components

| | |
|---|---|
| FK1 | 4-(2,5-diamino benzylamino)aniline*HCl |
| FK2 | 2-(3-aminophenyl)aminomethyl-1,4-diaminobenzene*HCl |

TABLE 5
Hair colorant

| | Example No. | | | | |
|---|---|---|---|---|---|
| | 1 | 2 | 3 | 4 | 5 |
| Dyes | (Quantity of dye in g) | | | | |
| 2-amino-5-ethylphenol | 0.30 | 0.30 | 0.30 | 0.30 | 0.03 |
| 3-amino-2,6-dimethylphenol | 0.002 | | | 0.03 | |
| 5-((2-hydroxyethyl)amino)- | | 0.002 | | 0.3 | 0.03 |
| 2-methylphenol | | | | | |
| 5-amino-2-ethylphenol | | | 0.002 | | 0.03 |
| Coloring results | bright | bright | bright | bright | bright |
| | yellow | yellow | yellow | yellow | yellow |
| | Example No. | | | | |
| | 6 | 7 | 8 | 9 | 10 |
| Dyes | (Quantity of dye in g) | | | | |
| 2-amino-5-ethylphenol | 0.30 | 0.30 | 1.0 | 0.5 | 0.8 |
| 3-amino-2,6-dimethylphenol | 0.03 | 0.02 | 0.7 | | |
| 5-((2-hydroxyethyl)amino)- | | 0.02 | | 0.4 | |
| 2-methylphenol | | | | | |
| 5-amino-2-ethylphenol | 0.03 | 0.02 | | | 0.6 |
| E10 | | | 0.5 | 0.3 | 0.4 |
| Coloring results | bright | bright | bright | bright | bright |
| | yellow | yellow | yellow- | orange | orange |
| | | | orange | | |
| | Example No. | | | | |
| | 11 | 12 | 13 | 14 | 15 |
| Dyes | (Quantity of dye in g) | | | | |
| 2-amino-5-ethylphenol | 0.002 | 0.005 | 0.02 | 0.08 | 0.32 |
| 3-amino-2,6-dimethylphenol | 0.3 | | | 0.002 | 0.02 |
| 5-((2-hydroxyethyl)amino)- | | 0.35 | | 0.001 | |
| 2-methylphenol | | | | | |
| 5-amino-2-ethylphenol | | | 0.005 | 0.003 | |
| E12 | 0.26 | 0.22 | 0.004 | 0.006 | |
| E14 | | | | | 0.02 |
| Coloring results | bright | bright | bright | bright | bright |
| | orange | orange | orange | orange | orange |
| | Example No. | | | | |
| | 16 | 17 | 18 | 19 | 20 |
| Dyes | (Quantity of dye in g) | | | | |
| 2-amino-5-ethylphenol | 0.025 | 0.042 | 0.1 | 0.07 | 0.05 |
| 3-amino-2,6-dimethylphenol | 0.01 | | | 0.02 | 0.001 |
| 5-((2-hydroxyethyl)amino)- | | 0.005 | | 0.010 | 0.002 |
| 2-methylphenol | | | | | |
| 5-amino-2-ethylphenol | | | 0.002 | 0.01 | 0.002 |
| E10 | | | | | 0.001 |
| E11 | | | | | 0.001 |
| E12 | | | | | 0.001 |
| E14 | 0.008 | 0.004 | 0.04 | 0.03 | 0.001 |
| Coloring results | bright | bright | bright | bright | bright |
| | orange | orange | orange | orange | orange |

| | Example No. | | | | |
|---|---|---|---|---|---|
| | 21 | 22 | 23 | 24 | 25 |
| Dyes | (Quantity of dye in g) | | | | |
| 2-amino-5-ethylphenol | 0.01 | 0.002 | 0.005 | 0.02 | 0.01 |
| 3-amino-2,6-dimethylphenol | 0.2 | | | 0.2 | 0.01 |
| 5-((2-hydroxyethyl)amino)- | | 0.3 | | 0.5 | |
| 2-methylphenol | | | | | |
| 5-amino-2-ethylphenol | | | 0.4 | 0.2 | |
| E15 | 0.25 | 0.45 | 0.6 | 1.0 | |
| E8 | | | | | 0.2 |
| Coloring results | violet | violet | violet | dark | violet |
| | | | | violet | |
| | Example No. | | | | |
| | 26 | 27 | 28 | 29 | 30 |
| Dyes | (Quantity of dye in g) | | | | |
| 2-amino-5-ethylphenol | 0.014 | 0.008 | 0.015 | 0.011 | 0.007 |
| 3-amino-2,6-dimethylphenol | | | 0.4 | 0.21 | |
| 5-((2-hydroxyethyl)amino)- | 0.4 | | 0.1 | | 0.25 |
| 2-methylphenol | | | | | |
| 5-amino-2-ethylphenol | | 0.25 | 0.1 | | |
| E8 | 0.3 | 0.2 | 0.5 | | |
| E9 | | | | 0.28 | 0.33 |
| Coloring results | violet | violet | dark | violet | violet |
| | | | violet | | |

| | Example No. | | | | |
|---|---|---|---|---|---|
| | 31 | 32 | 33 | 34 | 35 |
| Dyes | (Quantity of dye in g) | | | | |
| 2-amino-5-ethylphenol | 0.02 | 0.004 | 0.02 | 0.03 | 0.001 |
| 3-amino-2,6-dimethylphenol | | 0.002 | 0.01 | | |
| 5-((2-hydroxyethyl)amino)- | | 0.1 | | 0.01 | |
| 2-methylphenol | | | | | |
| 5-amino-2-ethylphenol | 0.5 | 0.4 | | | 0.015 |
| E9 | 0.03 | 0.9 | | | |
| E15 | | | 0.66 | 0.1 | 0.005 |
| K20 | | | 0.07 | | |
| K26 | | | 0.26 | 0.02 | |
| K27 | | | | | 0.06 |
| K30 | | | | | 0.03 |
| D3 | | | 0.9 | 0.1 | 0.02 |
| D4 | | | | | 0.01 |
| Coloring results | dark | dark | reddish | light | brownish |
| | violet | violet | brown | golden | light |
| | | | | blonde | blonde |

| | Example No. | | | | |
|---|---|---|---|---|---|
| | 36 | 37 | 38 | 39 | 40 |
| Dyes | (Quantity of dye in g) | | | | |
| 2-amino-5-ethylphenol | 0.025 | 0.001 | 0.053 | 0.0012 | 0.01 |
| 3-amino-2,6-dimethylphenol | 0.11 | | | 0.005 | 0.55 |
| 5-((2-hydroxyethyl)amino)- | | 0.03 | | 0.002 | |
| 2-methylphenol | | | | | |
| 5-amino-2-ethylphenol | | | 0.1 | 0.006 | |
| E8 | | | 0.03 | | |
| E9 | 0.61 | | 0.06 | 0.3 | |
| E15 | 0.7 | 0.058 | | 0.33 | 2.0 |
| E10 | 0.07 | | 0.351 | | |
| E12 | | | | | 0.1 |
| K12 | | 0.003 | | | |
| K13 | | | | | 0.1 |
| K20 | | | 0.005 | 0.006 | 0.5 |
| K26 | 0.005 | | 0.29 | 0.23 | 1.5 |
| K27 | 0.46 | | | 0.02 | |
| K30 | | | | | |
| D3 | | | 0.1 | | |
| Coloring results | brown | light blonde | light blonde | medium | dark |
| | mahagony | mahagony violet | reddish gold | blonde | brown |

| | Example No. | | | | |
|---|---|---|---|---|---|
| | 41 | 42 | 43 | 44 | 45 |
| Dyes | (Quantity of dye in grams) | | | | |
| 2-amino-5-ethylphenol | 0.02 | 0.01 | 0.015 | 0.01 | 0.001 |
| 3-amino-2,6-dimethylphenol | 0.1 | 0.2 | 0.05 | 0.1 | 0.002 |
| 5-((2-hydroxyethyl)amino)- | 0.4 | | 0.04 | 0.3 | 0.003 |
| 2-methylphenol | | | | | |
| 5-amino-2-ethylphenol | 0.7 | | 0.01 | 0.4 | 0.006 |
| E8 | 0.1 | | 3.0 | 0.2 | |
| E9 | 0.005 | 2.0 | 0.2 | 0.1 | |
| E15 | 2.5 | | 0.3 | 0.4 | 0.42 |
| E13 | 0.1 | 2.0 | 0.1 | | |
| K11 | | | 0.5 | | |
| K12 | 2.2 | | 0.11 | | 0.02 |
| K13 | 0.35 | | 0.13 | | 0.01 |
| K14 | | | 0.3 | | |
| K15 | 0.1 | | 0.2 | | |
| K16 | | | 0.015 | | |
| K17 | | | 0.2 | | |
| K18 | | | 0.1 | | |
| K19 | | | 0.01 | | |
| K20 | | 1.5 | 0.1 | | 0.08 |
| K24 | | | 0.5 | 0.1 | |
| K26 | | 2 | 1.3 | | 0.14 |
| Coloring results | blue- | black | black | violet | ash- |
| | black | | | | blonde |

TABLE 6
Hair colorant

| | Example No. | | | | |
|---|---|---|---|---|---|
| | 46 | 47 | 48 | 49 | 50 |
| Dyes | (Quantity of dye in grams) | | | | |
| 2-amino-5-ethylphenol | 0.5 | 0.02 | 0.025 | 0.1 | 0.02 |
| 3-amino-2,6-dimethylphenol | 0.1 | 0.04 | 0.01 | 0.3 | 1.0 |
| 5-((2- | 0.1 | 0.03 | 0.015 | 0.4 | 0.05 |
| hydroxyethyl)amino)- | | | | | |
| 2-methylphenol | | | | | |
| 5-amino-2-ethylphenol | 0.1 | | 0.01 | 0.4 | 0.05 |
| E12 | 1. | 0.45 | | 0.2 | 2.4 |
| E14 | 0.2 | | 0.1 | 1.8 | |
| E15 | 0.01 | 0.003 | 0.67 | 0.005 | |
| K12 | 0.001 | | | | 0.29 |
| K13 | 0.001 | 0.1 | | | 0.006 |
| K20 | 0.002 | 0.036 | 0.1 | | |
| K21 | 0.003 | | | 0.2 | |
| K22 | 0.002 | | | | |
| K23 | 0.001 | | | | |
| K24 | | | 0.009 | | |
| K26 | 0.02 | | 0.36 | | |
| K27 | 0.04 | | 0.003 | | |
| K28 | 0.02 | | | | |
| K29 | 0.02 | 0.292 | | | |
| K30 | 0.02 | | | | |
| Coloring results | red- | red-violet | dark | bright | crimson |
| | orange | blonde | blonde | red | red |

| | Example No. | | | | |
|---|---|---|---|---|---|
| | 55 | 56 | 57 | 58 | 59 |
| Dyes | (Quantity of dye in grams) | | | | |
| 2-amino-5-ethylphenol | 0.5 | 0.3 | 0.1 | 0.45 | 0.27 |
| 3-amino-2,6-dimethylphenol | 0.01 | | | 0.3 | 0.3 |
| 5-((2- | 0.2 | | 0.69 | 0.4 | 0.3 |
| hydroxyethyl)amino)- | | | | | |
| 2-methylphenol | | | | | |
| 5-amino-2-ethylphenol | 0.3 | 0.2 | | 0.4 | 0.3 |
| E10 | 0.8 | 2.3 | 1.9 | 0.92 | 1.2 |
| E15 | 0.2 | 0.002 | 1.3 | 0.2 | 0.6 |
| K31 | 0.01 | | | | |
| K32 | 0.002 | | | | |
| K33 | 0.002 | | | | |
| K34 | 0.2 | | | | |
| K35 | 0.1 | | | | |
| K36 | 0.002 | | | | |
| K37 | 0.02 | | | | |
| K38 | | | 0.01 | | |
| K39 | | | 0.02 | | |
| K40 | | | 0.7 | | 0.4 |
| K41 | | | 0.8 | | 0.4 |
| K42 | | 1.4 | 0.02 | 0.1 | |
| D3 | 0.3 | | | | 0.1 |
| Coloring results | orange- | bright | violet | golden | garnet |
| | red | orange | mahagony | red | red |

| | Example No. | | | | |
|---|---|---|---|---|---|
| | 60 | 61 | 62 | 63 | 64 |
| Dyes | (Quantity of dye in grams) | | | | |
| 2-amino-5-ethylphenol | 0.32 | 0.57 | 0.6 | 0.71 | 0.41 |
| 3-amino-2,6-dimethylphenol | 0.4 | 0.1 | 0.05 | 0.003 | 0.2 |
| 5-((2- | 0.1 | 0.4 | 0.1 | 0.2 | 0.2 |
| hydroxyethyl)amino)- | | | | | |
| 2-methylphenol | | | | | |
| 5-amino-2-ethylphenol | 0.1 | 0.1 | 0.05 | 0.015 | 0.2 |
| E10 | 0.2 | 0.2 | | 0.3 | |
| E12 | 0.2 | 0.2 | 0.1 | | |
| E14 | 0.1 | 0.2 | | | 0.5 |
| K27 | | 0.1 | | 0.005 | |
| K34 | | | | | 0.1 |
| D1 | 0.005 | | 0.2 | | |
| D2 | 0.02 | | | 0.01 | 0.2 |
| D3 | 0.3 | | | 0.2 | |
| D4 | | 0.1 | 0.3 | | |
| D5 | | 0.002 | 0.005 | 0.025 | |
| D6 | | 0.001 | 0.02 | 0.2 | |
| D7 | | 0.02 | | | |
| D8 | 0.1 | 0.3 | | | 0.3 |
| D9 | 0.002 | | | 0.1 | 0.2 |
| Coloring results | bright | bright red- | bright | bright | bright |
| | orange-red | orange | yellow | yellow- | red |
| | | | | orange | |

TABLE 7
Hair colorant

| | Example No. | | | | | | | |
|---|---|---|---|---|---|---|---|---|
| | 65 | 66 | 67 | 68 | 69 | 70 | 71 | 72 |
| Dyes | (Quantity of dye in grams) | | | | | | | |
| 2-amino-5-ethylphenol | 0.001 | 0.108 | 0.041 | 0.006 | 0.18 | 0.19 | 0.08 | 0.2 |
| 3-amino-2,6-dimethylphenol | 0.001 | 0.01 | 0.001 | 0.02 | 0.004 | 0.02 | 0.005 | 0.009 |
| 5-((2-hydroxyethyl)amino)- | 0.001 | 0.002 | 0.001 | 0.02 | 0.002 | 0.01 | 0.006 | 0.002 |
| 2-methylphenol | | | | | | | | |
| 5-amino-2-ethylphenol | 0.001 | 0.001 | 0.001 | 0.02 | 0.1 | 0.041 | 0.001 | 0.001 |
| E8 | | 0.12 | | 0.12 | | | | |
| E9 | | | 0.12 | | 0.005 | | | |
| E15 | | | | | | 0.13 | | |
| FK1 | 1.0 | | | 0.2 | | 0.38 | 0.38 | 0.38 |
| FK2 | | 0.45 | 0.07 | 02 | 0.58 | | | |
| Coloring results | deep | medium | medium | black- | brown | black- | medium | brown |
| | blue | brown | blonde | brown | | brown | brown | |

Unless otherwise indicated, all percent values in the present application refer to percent by weight.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An agent for oxidative coloring of keratin fibers, comprising a combination of 2-amino-5-ethylphenol and at least one m-aminophenol of general formula (I) 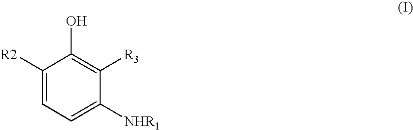
wherein R1=H or C₂H₄OH; R2=CH₃ or C₂H₅, and R3=H or CH₃;
wherein R2 is not equal to CH₃, if R1 and R3 equal H;
or physiologically compatible, water-soluble salts thereof.

2. An agent according to claim 1, wherein the m-aminophenol of general formula (I) is selected from the group consisting of 3-amino-2,6-dimethylphenol, 5-((2-hydroxyethyl)amino)-2-methylphenol and 5-amino-2-ethylphenol.

3. An agent according to claim 1, wherein the total quantity of agent comprises from 0.001% to 5% by weight of 2-amino-5-ethylphenol.

4. An agent according to claim 1, wherein the total quantity of agent comprises from 0.001% to 5% by weight of the m-aminophenol of general formula (I).

5. An agent according to claim 1, further comprising additional conventional dyes, which are selected from the group consisting of oxidative dyes, natural direct dyes, nature-identical direct dyes, and synthetic direct dyes.

6. An agent according to claim 1, wherein said agent is a hair colorant.

7. A two-component agent for oxidative coloring of keratin fibers consisting of a component A and an oxidizing-agent-containing component B, wherein component A comprises a combination of 2-amino-5-ethylphenol and at least one m-aminophenol of general formula (I) 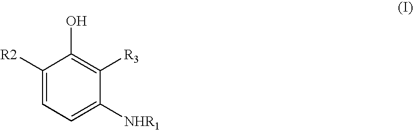
wherein R1=H or C₂H₄OH; R2=CH₃ or C₂H₅, and R3=H or CH₃;
wherein R2 is not equal to CH₃, if R1 and R3 equal H;
or physiologically compatible, water-soluble salts thereof.
